(19) United States
(12) Patent Application Publication
(10) Pub. No.: US 2025/0235141 A1
(43) Pub. Date: Jul. 24, 2025

(54) MICROELECTRODE GRID WITH FLAP FOR CONTINUOUS INTRAOPERATIVE NEUROMONITORING

(71) Applicant: THE REGENTS OF THE UNIVERSITY OF CALIFORNIA, Oakland, CA (US)

(72) Inventors: Shadi A. DAYEH, San Diego, CA (US); Youngbin TCHOE, San Diego, CA (US); Ahmed M. RASLAN, Portland, OR (US)

(21) Appl. No.: 18/832,850

(22) PCT Filed: Feb. 3, 2023
(86) PCT No.: PCT/US2023/061906

§ 371 Date: Jul. 24, 2024

Related U.S. Application Data
(60) Provisional application No. 63/306,545, filed on Feb. 4, 2022.

(51) Int. Cl. A61B 5/294; A61B 5/268; A61B 5/388

(52) U.S. Cl. CPC A61B 5/294; A61B 5/268; A61B 5/388; A61B 2562/164

(57) ABSTRACT

A microelectrode grid for continuous interoperative neuromonitoring includes a flexible substrate and a plurality of low impedance electrochemical interface materials on conducting metal pads on the substrate. The metal pads are interconnectable to stimulation/acquisition electronics through metal lead interconnects forming stimulation and recording channels and eventually to bonding pads. The interconnects are insulated with dielectric. A flap within the substrate is movable away from the remainder of the substrate while at least some of the metal pads on the remainder of the substrate can remain in contact with an organ when the flap is moved away from the remainder of the substrate.

### PRIORITY CLAIM AND REFERENCE TO RELATED APPLICATION

The application claims priority under 35 U.S.C. § 119 and all applicable statutes and treaties from prior U.S. provisional application Ser. No. 63/306,545, which was filed Feb. 4, 2022.

### STATEMENT OF GOVERNMENT INTEREST

This invention was made with government support under grant numbers CMMI-1728497 awarded by National Science Foundation and grant number DP2-EB029757 awarded by the National Institutes of Health. The government has certain rights in this invention.

### FIELD

A field of the invention is electrophysiology from the nervous system. The invention provides a microelectrode grid, which has application as an electrophysiological grid.

### BACKGROUND

Electrocorticography (ECoG) grids measure electrophysiological activity from the surface of the brain and are conventionally used before resecting the brain tissues to delineate the functional and diseased boundaries. Together with the pre-surgical functional MRI (fMRI), the ECoG functional mapping is a powerful tool that neurosurgeons use to distinguish pathological tissues from healthy tissues and determine the resection boundary. However, the conventional ECoG grid does not provide continuous intraoperative neuromonitoring (cIONM) because the electrode blocks the surgical field.

cIONM is used in spinal cord surgeries where electrical pulses applied to the peripheral nerves result in somatosensory evoked potentials (SSEPs) that are measured by electroencephalography (EEG) electrodes from the surface of the scalp. Though these measurements aim to ensure connection pathways through the spinal cord and are indirect measurements of the health status of the spinal cord, the amplitude of the SSEPs inform the surgeon on the progress of the surgery to avoid postoperative deficits. The application of electrophysiological grids to the surface of the spinal cord enable better and more accurate assessment of its health during surgery and can allow establishing the midline, an anatomical landmark that is crucial for guiding spinal surgery. Despite its advantage in preserving essential nerves, cIONM is effective in preserving essential nerves and is used in a handful of surgeries involving the nervous system. In addition to spinal cord surgery, cIONM is used in thyroid surgery (to prevent recurrent laryngeal nerve iatrogenic damages by monitoring vagal nerve) or cerebellopontine angle surgery (facial nerve monitoring). The current practice of cIONM works by manually placing a few electrodes onto the nerves to continuously stimulate or record from them. cIONM has been available for thyroid surgery, surgery on the posterior cranial fossa, and surgery on vascular anomalies where vagus, facial, and vestibulocochlear nerves are at risk. The individual electrodes used for nerve stimulation or recording are manually placed on relevant nerves that should be preserved during surgery. The individual electrodes are generally designed to preserve nerve bundles and are usually composed of single channel that is incapable of doing spatial mapping of nerve system as complicated as brain mapping.

Adtech Medical, Natus, and Integra all commercialize ECoG grids with different contact arrangements. The contacts are generally made of Pt, PtIr, or SST, and are pressed in a silicone film that is about 0.5 mm thick. Contacts are typically spaced at a 1 cm pitch, though custom-made higher density grids (256 channels, 4 mm pitch, 1.17 mm electrode diameter, coverage area of 6.4 cm×6.4 cm) have recently become available.

Blackrock Microsystems is also commercializing ECoG arrays that, according the company's website, are up to 50% thinner than comparable products, require a fewer number of cables, and have variable diameter sizes from 0.3 mm-0.7 mm. The limitations of these handmade grids with contact pressing and wire soldering on the back of the contact are well known to limit their scalability to large numbers of contact and to tight contact spacing.

NeuroOne Inc. offers a thin-film cortical electrode technology that is built on top of 25 μm thick polyimide layers. The cortical electrode is offered under the Evo® 7 tradename. It includes a single thin tail that allows the implanted electrode tail to be tunneled through one incision and connects to a disposable cable assembly. Various electrode configurations are offered, with an example having two rows of parallel electrodes. This technology permits pre- and post-surgical stimulation and monitoring but can't provide continuous monitoring of a surgical site during surgery.

Existing approaches for increasing the channel count involved the integration of integrated circuits on the electrodes to facilitate wiring and reduce the wire count from the electrode itself to the outer world. Transfer of Si transistors to flexible substrates is one solution to increase integration.

Neuralink has successfully adopted conventional flip-chip bonding techniques to form interconnects between custom CMOS chips and polymer-based electrode threads in penetrating electrode grid designs. Similar penetrating electrode designs are described in Melosh et al., WO2018183967; US20160128588; and WO2019173572. While penetrating electrodes can be used for IONM, surgeons prefer to avoid brain penetration during surgical resection to avoid unnecessary brain tissue damage. These type of grids are better suited for deep-brain recording and stimulation.

Dr. Langer Medical produces a single channel Saxophone® electrode that wrap around the vagus nerve to prevent recurrent laryngeal nerve damage during the thyroid surgery. The Saxophone® electrode can continuously stimulated the vagus nerve outside the immediate operation site for additionally safety. Dr. Langer Medical also produces single channel thin tube electrode to record signal from vocalis muscle to monitor the perseverance of the nerves during the surgery

Medtronic also provides the Automatic Periodic Stimulation™ (APS) continuous monitoring electrode together with a NIM™ nerve monitoring system to enable early detection and warning of a change in nerve function during the thyroid surgery. This cIONM allows the surgeon to take immediate corrective action to prevent potential injury. The APS™ electrode is placed on the vagus nerve and delivers continuous low-level stimulation. A baseline of nerve function is obtained, and subsequent EMG responses are monitored and charted in real time to provide feedback. The APS continuous monitoring electrode is also composed of single channel stimulation/recording electrodes.

### BACKGROUND PUBLICATIONS

1. P. Stankovic, J. et al, “Continuous intraoperative neuromonitoring (cIONM) in head and neck surgery-a review,” HNO 68, 86 (2020).
2. Hermiz, J. et al, “Sub-millimeter ECoG pitch in human enables higher fidelity cognitive neural state estimation,” NeuroImage 2018, 176, 454-464.
3. Duffau, H. et al, “Intra-operative direct electrical stimulations of the central nervous system: the Salpetriere experience with 60 patients,” Acta neurochirurgica 1999, 141 (11), 1157-1167.
4. Ganji, M., et al., “Selective formation of porous Pt nanorods for highly electrochemically efficient neural electrode interfaces. Nano letters,” 19(9), 6244-6254.
5. Viventi, J. et al., “Flexible, foldable, actively multiplexed, high-density electrode array for mapping brain activity in vivo,” Nature neuroscience 2011, 14 (12), 1599.
6. Fang, H. et al, “Capacitively coupled arrays of multiplexed flexible silicon transistors for long-term cardiac electrophysiology,” Nature biomedical engineering 2017, 1 (3), 0038.
7. Shokoueinejad, M. et al, “Progress in the Field of Micro-Electrocorticography,” Micromachines 2019, 10 (1), 62.
8. Kim, D.-H. et al., “Flexible and stretchable electronics for biointegrated devices,” Annual review of biomedical engineering 2012, 14, 113-128.
9. Won, S. M. et al., “Recent Advances in Materials, Devices, and Systems for Neural Interfaces,” J. A., Advanced Materials 2018, 30 (30), 1800534.
10. Wellman, S. M. et al., “A Materials Roadmap to Functional Neural Interface Design,” Advanced functional materials 2018, 28 (12), 1701269.
11. Robinson, J. T. et al., “Developing Next-generation Brain Sensing Technologies—A Review,” IEEE Sensors Journal 2019.
12. Hong & Lieber, “Novel electrode technologies for neural recordings,” Nature Reviews Neuroscience 2019, 1.
13. Williams, J. C. et al., U.S. Pat. No. 8,386,007, “Thin-film micro electrode array and method.”
14. Williams, J. C. et al., U.S. Pat. No. 8,483,794, “Method for implanting an electrode that unfurls in response to a predetermined stimulus.”
15. Khodagholy, D. et al. “NeuroGrid: recording action potentials from the surface of the brain,” Nature neuroscience 2015, 18 (2), 310-315.
16. Khodagholy, D. et al., “Organic electronics for high-resolution electrocorticography of the human brain,” Science advances 2016, 2 (11), e1601027.
17. Uguz, I. et al., “Autoclave Sterilization of PEDOT:PSS Electrophysiology Devices,” Advanced healthcare materials 2016, 5 (24), 3094-3098.
18. Ganji, M. et al., “Development and Translation of PEDOT:PSS Microelectrodes for Intraoperative Monitoring,” Advanced Functional Materials 2018, 28 (12), 1700232.
19. Ganji, M. et al., “Strong adhesion of wet conducting polymers on diverse substrates,” Advanced healthcare materials 2018, 7 (22), 1800923.
20. Viventi, J. et al., “Flexible, foldable, actively multiplexed, high-density electrode array for mapping brain activity in vivo,” Nature neuroscience 2011, 14 (12), 1599-1605.
21. Viventi, J. et al., “A conformal, bio-interfaced class of silicon electronics for mapping cardiac electrophysiology,” Science translational medicine 2010, 2 (24), 24ra22-24ra22.
22. Kim, D.-H. et al., “Dissolvable films of silk fibroin for ultrathin conformal bio-integrated electronics,” Nature materials 2010, 9 (6), 511.
23. E. Musk, “An integrated brain-machine interface platform with thousands of channels,” Journal of medical Internet research 21, e16194 (2019).
24. A. Obaid et al., “Massively parallel microwire arrays integrated with CMOS chips for neural recording,” Science advances 6, eaay2789 (2020).
25. Zhang et al., U.S. Pat. No. 7,905,013: Method for forming an iridium oxide (IrOx) nanowire neural sensor array.

### SUMMARY OF THE INVENTION

A preferred embodiment provides a microelectrode grid for continuous interoperative neuromonitoring. The microelectrode grid includes a flexible substrate and a plurality of low impedance electrochemical interface materials on conducting metal pads on the substrate. The metal pads are interconnectable to stimulation/acquisition electronics through metal lead interconnects forming stimulation and recording channels and eventually to bonding pads. The interconnects are insulated with dielectric. A flap within the substrate is movable away from the remainder of the substrate while at least some of the metal pads on the remainder of the substrate can remain in contact with an organ when the flap is moved away from the remainder of the substrate.

### BRIEF DESCRIPTION OF THE DRAWINGS

FIGS. 1A-1C are schematic respective front view, backside view and backside view with flap open diagrams of a preferred microelectrode grid for continuous interoperative neuromonitoring;

FIG. 2 is flowchart of a preferred method for interoperative neuromonitoring using a preferred microelectrode grid for continuous interoperative neuromonitoring;

FIGS. 3A-3D are images showing use of a preferred microelectrode grid for continuous interoperative neuromonitoring; and

FIGS. 3E-3F are images showing a preferred microelectrode grid for continuous interoperative neuromonitoring applied to an organ in respective flap-closed and flap open states.

### DETAILED DESCRIPTION OF THE PREFERRED EMBODIMENTS

Preferred embodiment microelectrode grids include a flap within a substrate that carries metal pads. The flap is movable away from the remainder of the substrate while at least some of the metal pads on the reminder of the substrate can remain in contact with an organ when the flap is moved away from the remainder of the substrate. This can greatly improve the ECoG neuromonitoring practice by allowing cIONM while functioning as conventional ECoG grid and recording in the regions surrounding, and if desired within, the resected tissue. Preferred embodiment microelectrode grids can provide cIONM for any part of the brain or spinal cord surface with channel counts up to thousands of channels that can be distributed based on the patient's specific indication and anatomy. Real-time feedback from thousands of channels can provide rich information to a neurosurgeon, especially when operating on a highly sensitive and sophisticated region of the brain. Tissue resection can be conducted with flap open. After the tissue resection is complete, the opened flap in the grid can be closed back, which permits post-surgical ECoG mapping, which can function to instantly provide information of the surgical outcome.

A preferred embodiment provides an electrophysiological grid, a flap part of which can be displaced from the tissue while the other parts of the grid remain in intimate contact with tissue. A foldable flap structure allows part of the grid to be flipped back away from tissue and then placed back on tissue when needed. This approach enables the continuous intraoperative neuromonitoring (cIONM) of the brain or spine state and their activity during the resective neurosurgery. The flap located on the grid can be opened and closed, allowing the surgical tools to access and resect the brain or spine tissue through the inner window of the microelectrode. The surrounding microelectrode recording channels outside the circular flap region are capable of continuously monitoring the electrophysiological activities during the entire neurosurgery. The capability to do cIONM and provide live feedback to the neurosurgeon are crucial in preserving essential functions on the human brain and spinal cord and may improve patient outcome.

Preferred embodiments of the invention will now be discussed with respect to experiments and drawings. Broader aspects of the invention will be understood by artisans in view of the general knowledge in the art and the description of the experiments that follows.

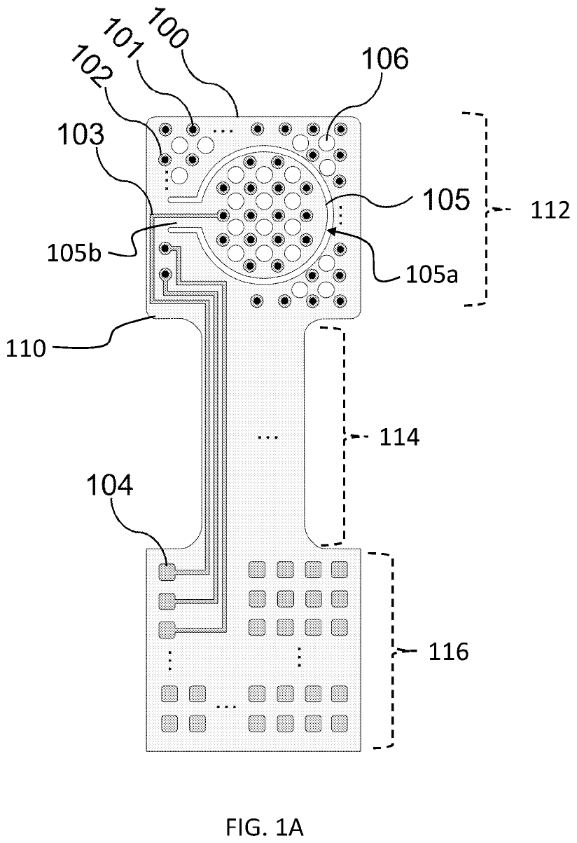

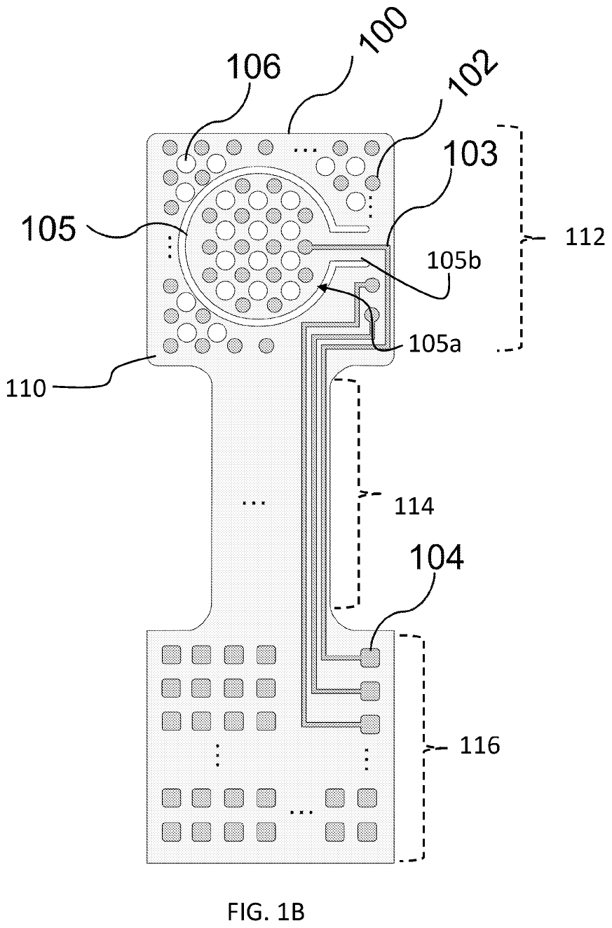

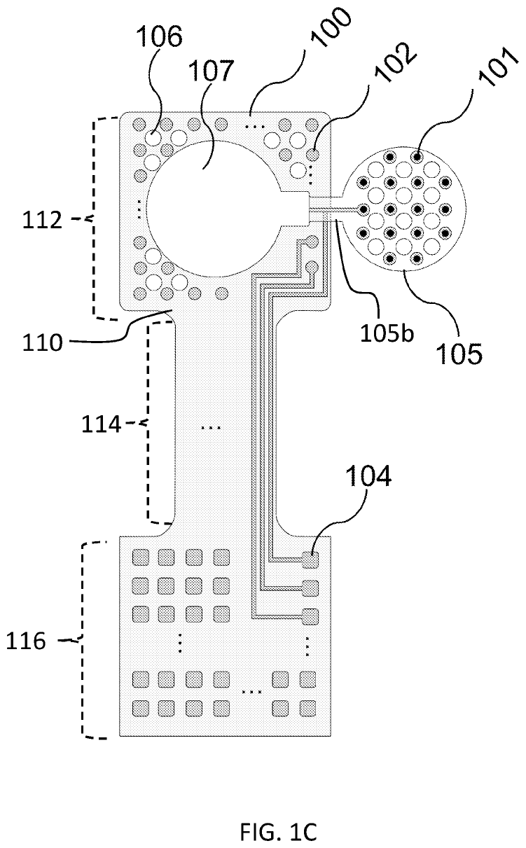

FIGS. 1A-1C illustrate a preferred microelectrode (μECoG) grid 100. FIG. 1A shows the front side with a flap 105 closed and showing the stimulation/recording sites. FIGS. 1B and 1C show the backside respectively with the flap 105 closed and open. The grid includes the flap (e.g., circular, while other shapes can be used) 105 within the grid (e.g., near the center of the grid, FIGS. 1A and 1B).

The μECoG grid 100 includes a plurality of stimulation/recording sites formed of low impedance electrochemical interface materials 101 on conducting metal pads 102. The metal pads can be individually connected to stimulation/acquisition electronics (not shown) through separate, individual metal lead interconnects 103 that terminate to separate individual bonding pads 104. Only three interconnects 103 are illustrated for simplicity's sake, while artisans will appreciate the each conducting metal pad 102 and each bonding pad 104 is connected by an individual metal lead interconnect 103. Numbers of conducting metal pads 102, interconnects 103 and bonding pads 104 can have a pitch as small as 10 μm, and thereby allow thousands of separate channels. The technique for forming the encapsulated array μECoG grid 100 is the same as a flat continuous grid disclosed in Dayeh et al., PCT/US22/19778, entitled Multi-Hundred or Thousand Channel Electrode Electrophysiological Array and Fabrication Method. Electrode densities and numbers of channels providing by the conducting metal pads can be in the hundreds or thousands as in PCT/US22/19778. Most of an outer portion 105a of the flap 105 is etched through to permit the flap 105a to be folded back at a hinge area 105b of the flap 105. The pattern is established such that conducting metal pads that are within the flap 105 have their interconnects routed through the hinge area 105b of the flap. Other interconnects 103 are routed around the flap portion, and their associated conducting metal pads 102 therefore can provide signals when the flap 105 is open.

The entire metal leads are encapsulated with thin and freestanding biocompatible polymer layers 110. Through holes 106 formed throughout the polymer layers 110 at a sensing region 112 of the μECoG grid 100 to achieve intimate contact between the μECoG grid and an organ, e.g., brain surface. Both the inner circular flap region 105 as well as the outer region of the μECoG grid contain recording sites 101, and the circular flap region 105 can work in either close or open configuration. When the circular flap is in the open configuration (FIG. 1C), surgical tools can access the brain, spinal, or cardiac tissues through the circular window while the outer region could keep doing the cIONM throughout the entire resective neurosurgery.

The polymer layers 110 form a flexible, unitary carrier that defines the sensing portion 112, which is applied to an organ. The sensing portion 112 is sized according to the surgical procedure. For example, a small 1×1 cm² sensing portion 112 can be appropriate in the context of the spinal cord, while larger sensing portions, e.g. 8×8 cm² for the brain. The remainder of the carrier includes a neck portion 114, which is preferably narrower than the sensing portion 112 and can be sized to insert through a small incision. The remainder of the unitary carrier forms a circuit connection portion 116, which is sized and shaped to bond to an external stimulation/acquisition electronics. The neck portion 114 can be much longer than either of the sensing 112 or circuit connection portions 114. The neck portion 114 is preferably long enough to extend 3-10 cm or more away from the sensing portion 112. Generally, the distance the neck portion extends is preferred to be longer, and distance of 30 cm or more can be used. Generally, the distance the neck portion 114 extends provides sufficient clearance for a surgeon to operate without impedance from electronics connected to the circuit connection portion 116. The sensing portion 112 and adjacent portion of the neck portion 114 will be packaged to be sterile.

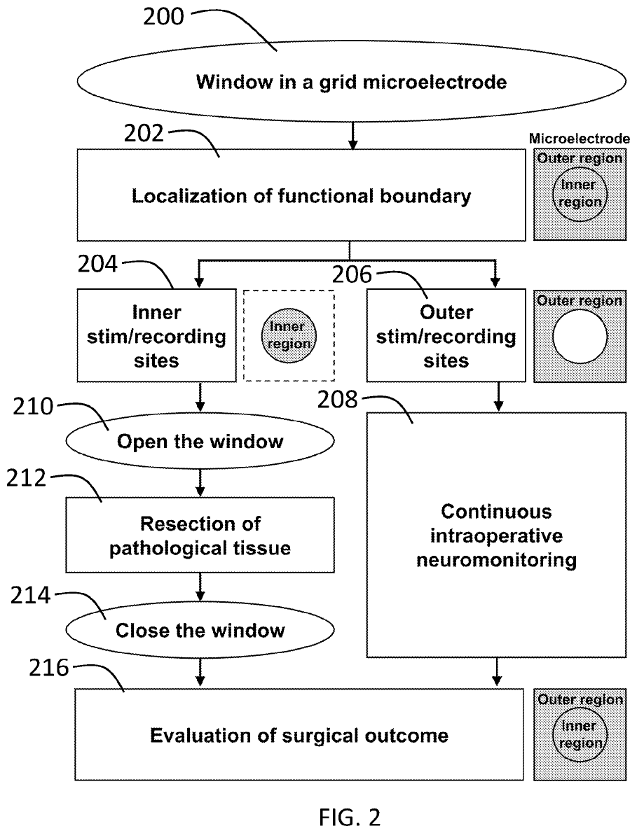

FIG. 2 illustrates control of a preferred μECoG during surgery. An μECoG of the invention is provided. A functional boundary area is localized 202 by measuring electrophysiological activity on the μECoG in response to task such as auditory stimuli, speech, movement, or a memory task. Electronics connected to the circuit connection portion 116 then record or stimulate the inner contacts (within the closed flap) 204 and outer contacts (outside of the closed flap) 206. All contacts can be monitored after stimulation, and the outer contacts continue stimulation/monitoring 208 while the flap is lifted to open a surgical window 210, a surgeon performs resection or any other surgical procedure through the window 212, and then closes the flap to close the surgical window 214. Upon closure of the surgical window 214, the stimulation/monitoring 208 then resumes for both the inner recording sites (within the closed flap) outer contacts (outside of the closed flap). Evaluation 216 then can be conducted by the electronics and/or a health care professional reviewing data provided through display or other output format by the electronics connected to the circuit connection portion 116, providing immediate post-operative information and information obtained during surgery.

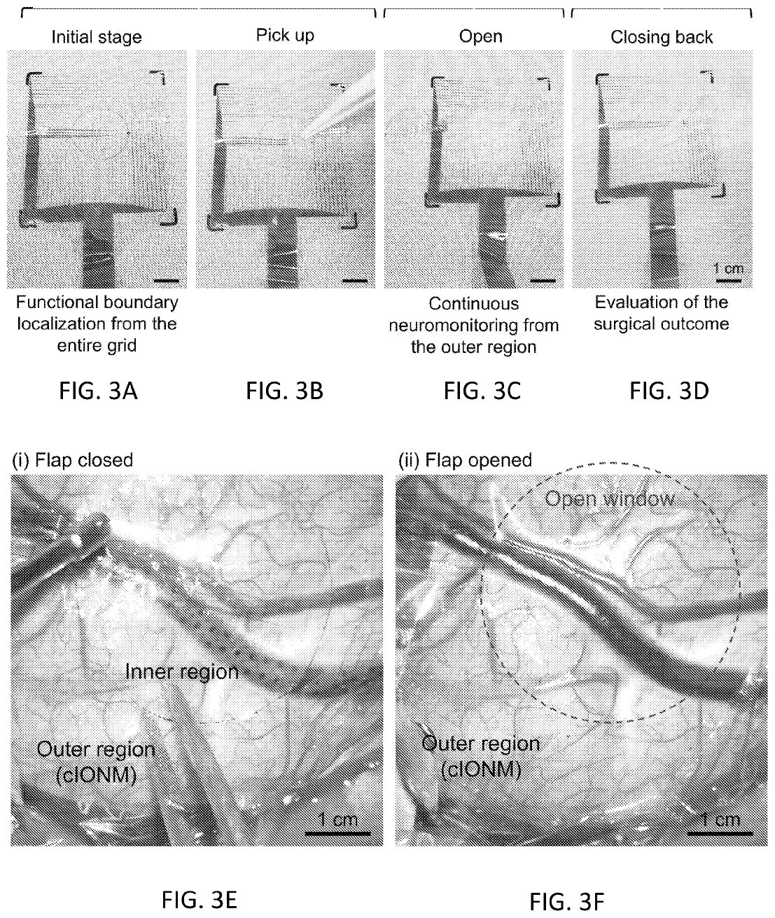

A prototype μECoG device was fabricated in accordance with the invention and was tested. The μECoG grid with a circular flap and its use is shown by photos (FIGS. 3A-3D). Actual placement of the electrode on a human brain with open and close configurations was demonstrated (FIGS. 3E and 3F). The prototype dimensions were 5 cm×5 cm. The neck portion extended 3 cm. The flap portion was 2.6 cm in diameter and had 250 conducting metal pads providing 250 channels. There were 750 conducting pads outside of the flap portion providing 750 channels. Signals were acquired via Intan 1024 channel recording controller. The prototype was sterilized by V-PRO® and sterile packaging was Duraholder® pouches.

The invention enables cIONM during surgical resection operations where the neurosurgeon will be able to monitor the functional mapping of brain, spine, or heart surface in real-time to correct their procedures immediately. This can greatly enhance the safety and outcome of resective neurosurgery. In addition to the brain and spine surgery, this invention can be used in general tumor surgery that involves nerves that need to be preserved, including thyroid surgery and other organs in the body.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A microelectrode grid for continuous interoperative neuromonitoring:
a flexible substrate;
a plurality of low impedance electrochemical interface materials on conducting metal pads on the substrate, the metal pads being interconnectable to stimulation/acquisition electronics through metal lead interconnects forming stimulation and recording channels and eventually to bonding pads, the interconnects being insulated with dielectric; and
a flap within the substrate, the flap being movable away from the remainder of the substrate while at least some of the metal pads on the remainder of the substrate can remain in contact with an organ when the flap is moved away from the remainder of the substrate.

2. The microelectrode grid of claim 1, wherein the remainder of the substrate comprises surrounding microelectrode recording channels outside the flap, the surrounding microelectrode channels being capable of continuously monitoring during surgery.

3. The microelectrode grid of claim 1, wherein the dielectric comprises biocompatible polymer layers.

4. The microelectrode grid of claim 3, wherein the dielectric encapsulates the metal lead connects.

5. The microelectrode grid of claim 1, wherein the flap comprises a hinge and the metal lead interconnects for the at least some of the metal pads are routed through the hinge.

6. The microelectrode grid of claim 1, wherein the substrate and dielectric are unitary flexible polymer that encapsulates the metal lead electrodes.

7. The microelectrode grid of claim 6, wherein the unitary flexible polymer defines a sensing portion including the conducting metal pads, the flap and portions of the metal lead interconnects, a neck portion that extends away from the sensing portion, and a circuit connection portion that includes the bonding pads.

8. The microelectrode grid of claim 7, wherein the sensing portion comprises through-holes for intimate contact with an organ during surgery.

9. The microelectrode grid of claim 7, wherein the neck portion is narrower than both of the sensing portion and the circuit connection portion.

10. The microelectrode grid of claim 9, wherein the neck portion has a length to extend the circuit connection portion away from a surgical site and permit a surgeon to operate without impedance from electronics connected to the circuit connection portion.

11. The microelectrode grid of claim 1, wherein the flap is large enough to provide a surgical access site accessible with surgical tools.
